(19) United States
(12) Patent Application Publication
(10) Pub. No.: US 2010/0106192 A1
(43) Pub. Date: Apr. 29, 2010

(54) System and method for aligning vertebrae in the amelioration of aberrant spinal column deviation condition in patients requiring the accomodation of spinal column growth or elongation

(76) Inventor: Mark A. Barry, Las Vegas, NV (US)

Correspondence Address: PATTON BOGGS LLP, 8484 WESTPARK DRIVE, SUITE 900, MCLEAN, VA, 22102, US

(21) Appl. No.: 12/458,948

(22) Filed: Jul. 28, 2009

Related U.S. Application Data
(63) Continuation-in-part of application No. 12/258,488, filed on Oct. 27, 2008.

(51) Int. Cl. A61B 17/70; A61B 17/88

(52) U.S. Cl. 606/258; 606/264; 606/279

(57) ABSTRACT

A system and method for ameliorating spinal column anomalies, such as scoliosis, while accommodating growth of juvenile patients, which include pedicle screws and an extendable telescopic spinal rod of non-circular cross section. Each pedicle screw includes spinal rod engagement means of complementary shape to the spinal rod for allowing longitudinal movement of the spinal rod, while resisting axial rotation of the pedicle screw relative to the spinal rod. The spinal rod is thereby allowed to slide longitudinally as attached vertebrae move during growth, while movement in other directions is arrested to preserve a proper orientation of involved vertebrae and to maintain scoliotic correction in three dimensions.

### CROSS-REFERENCE TO RELATED APPLICATIONS

This application is a continuation-in-part of U.S. patent application Ser. No. 12/258,488 filed Oct. 27, 2008; the content of which is incorporated by reference in its entirety.

### BACKGROUND OF THE INVENTION

1. Field of The Invention

The present invention relates to methods and apparatus for management and correction of spinal deformities, such as scoliosis.

2. Background Information

A serious deficiency presently exists with respect to conventional treatment and instrumentation for treating spinal deviation anomalies (such as scoliosis). This is particularly true as relates to juvenile cases involving greater than 45° curvatures (as such terminology is understood in the field) and more particularly to idiopathic scoliosis.

Currently, idiopathic scoliosis (“I.S.”) comprises approximately 75% of all juvenile cases. Those I.S. cases involving curvatures in the 25° - 45° range indicate treatment through bracing (beginning roughly at the bottom end of this range), but become untreatable by bracing (roughly at the top end of this range). Curvatures in excess of 45° indicate surgical intervention.

Use of implanted spinal rod systems of the current art introduces significant patient risks. These risks include considerable likelihood of hardware dislodgement (such as when hooks are used to engage spinal rod system components), ulcerations of skin that overlies protrusions of implanted systems, premature fusion of adjacent vertebrae with highly deleterious growth and spinal contour issues, impairment of longitudinal spinal growth, worsening of axial plane deformities such as rib hump, aggravation of truncal balance problems, and greater chance of infections.

To make matters worse, existing spinal rod systems, particularly when used in juveniles, require periodic lengthening and adjusting to accommodate growth (roughly every 9-18 months). For growing patients, especially juveniles, periodic lengthening and adjusting accommodates the change or increase in distance between spinal segments. Multiple surgical procedures may be required to adjust one or more components for lengthening and adjusting the spinal device. Further still, the existing systems only control curvature in two dimensions. Finally, a formal fusion procedure is required at or near skeletal maturity.

An ideal system for addressing the present shortcomings of treatment options for juvenile scoliosis involving greater than 45° curvatures is one which (at least): (1) provides three-dimensional correction of spinal anomalies; (2) provides secure engagement between instrumentation of affected vertebrae; (3) obviates or diminishes the need for periodic lengthening procedures; and (4) obviates the need for formal fusions at skeletal maturity.

Such a system would only be possible were it to “grow” with the patient (accommodate changes in distance in spinal segments or vertebrae), utilize other than easily dislodgeable skeletal engagement means, and maintain desired orientation and alignment of vertebrae in all dimensions.

With respect to this latter objective: current spinal rods are of circular or round cross section. Were present spinal rods or attachment means to be left “loose” to accommodate longitudinal motions as vertebrae move relatively as a result of growth, there would be nothing to combat the axial rotation of the vertebrae (relative to the spinal rod) even as they are constrained in their longitudinal movement along the rod. Such axial rotation would result in far less than optimal correction of the overall spinal topography.

Were an ideal system for addressing juvenile scoliosis requiring surgical intervention to become available (addressing each of the above-listed shortcomings of the systems and methods of the present art), the recipients would benefit in at least the following ways: (1) they would enjoy a much higher incidence and degree of success in alleviating their spinal deformities (in all dimensions of spinal column topography); (2) they would achieve more nearly normal growth expectations; (3) they would be spared from multiple surgical procedures with their associated risks; (4) they would not face the painful and potentially catastrophic consequences of spinal rod system component dislodgement; and (5) they would maintain mobility at adulthood that would otherwise be lost though otherwise required fusions.

### SUMMARY OF THE INVENTION

In view of the foregoing, it is an object of certain embodiments of the present system to provide an improved system of spinal instrumentation for use in ameliorating aberrant spinal column deviation conditions, such as scoliosis, particularly (though not necessarily solely) in juvenile cases of idiopathic scoliosis.

It is another object of certain embodiments of the present system to provide an improved system and associated method for ameliorating aberrant spinal column deviation conditions, such as scoliosis, which system and method addresses each of the above-listed shortcomings of the spinal rod systems and methods for addressing juvenile scoliosis that is of the present art.

It is another object of certain embodiments of the present system to provide an improved system and associated method for ameliorating aberrant spinal column deviation conditions, such as scoliosis, which system and method reduce hazards to patients relating at least to implantation of instrumentation, subsequent post-implantation surgical interventions related to accommodation of patient growth, spontaneous vertebral fusions, and inhibition of normal growth of the spine.

It is another object of certain embodiments of the present system to provide an improved method for ameliorating aberrant spinal column deviation conditions, such as scoliosis, which system accommodates growth without surgical intervention to the degree required of spinal rod systems of the present art.

It is another object of the present system to provide an improved system of spinal instrumentation, and a method for the use thereof, for ameliorating aberrant spinal column deviation conditions, such as scoliosis, which system and method facilitate maintaining spinal correction in three dimensions, rather than the mere two dimensions presently achievable (to a limited degree, and with limited success) with systems and methods of the present technology.

In satisfaction of each of the stated objects, as well as objects of natural extension thereof, embodiments of the inventor's present system provide an improved system and method for use of such system which will afford its recipients with one or more of the following benefits: (1) a much higher incidence and degree of success in alleviating their spinal deformities (in all dimensions of spinal column topography); (2) achievement of more nearly normal growth expectations; (3) the avoidance of multiple surgical procedures and associated discomfort and risks otherwise required in association with presently available spinal rod systems; (4) the elimination of substantially all risk of spinal rod system component dislodgement; and (5) the maintenance of mobility at adulthood that would otherwise be lost though otherwise required fusions.

The spinal rod system and the method for use described herein, of which is intended primarily to treat cases of juvenile scoliosis involving curvatures of greater than 45°, includes, in summary, an adjustable length spinal rod, specifically an extendable telescopic spinal rod with a means to slide or pass one end within another longitudinally, an anchor, for example a bone screw, more particularly a pedicle screw having a segment to be engaged to a bone or vertebra and a head segment to interface with the spinal rod, and a securing means configured for securing a mechanical engagement between the pedicle screw, for example, and the spinal rod for slidably engaging a spinal rod of non-circular cross section to facilitate longitudinal, patient growth-related movement of the pedicle screws relative with associated vertebrae. Because of the complimentary contours of the non-circular spinal rods and the extendable telescopic spinal rod, a “slide-only engagement” is achieved. That is to say that longitudinal movement of the pedicle screws with the vertebrae is allowed, particularly during growth, while at the same time axial rotation and other undesirable movement of the pedicle screw heads relative to the spinal rod (and the relative motion of the attached vertebrae) is substantially or nearly, completely arrested. Therefore, once the spinal rod is itself contoured according to the desired spinal topography, optimal scoliotic correction (in three dimensions) is achieved, not only at the time of initial implantation, but is perpetuated as the patient grows. Further or future spinal longitudinal growth is modulated by control in three dimensions. As used herein, reference to “extendable” is meant to be the common definition, for example lengthening, elongating, or stretching. Also, telescopic or telescoping is meant to be slide or pass one within another or lengthwise movement with one part entering another as the result of elongating or compressing or one part sliding alongside the other part, for example as a U-shaped rod resting within another U-shaped rod.

Optimal methods for achieving the initial scoliotic correction in three dimensions, which the present invention will maintain for the growing (juvenile) patient are best illustrated through reference to U.S. Patent Application, Publication No. 2006/0195092, which Application (and resulting Patent, if any) is hereby incorporated by reference.

### BRIEF DESCRIPTION OF THE DRAWINGS

The present invention may be more easily understood with reference to figures, which are as follow:

FIG. 1 is a diagrammatic, dorsal view of a spinal column with a growing spinal rod system of the present invention attached to selected vertebrae thereof.

FIG. 2 is a perspective depiction of an example of a pedicle screw having the unique spinal rod engagement means of the present invention for preventing axial rotation of the pedicle screw (and associated vertebrae) relative to the spinal rod.

FIG. 3 is a diagrammatic side elevational view of a spinal column having the preferred three pedicle screw “clusters” situated for engaging a spinal rod for practice of the method of the present invention.

FIG. 4A is a diagrammatic, perspective view of the extendable telescopic spinal rod of the present growing rod spinal deviation correction system, shown engaged with pedicle screw anchors as a non-circular cross-sectional spinal rod in the “slide-only engagement” as an unextended position. FIG. 4B is a perspective view of the extendable telescopic spinal rod of the present invention in an elongated position, which is achievable in the present growing rod spinal deviation correction system.

FIG. 5A is a diagrammatic, perspective view of the extendable telescopic spinal rod of the present invention, shown as a square geometry for axial plane control of the growing rod spinal deviation correction system. FIG. 5B is a perspective view of the extendable telescopic spinal rod of the present invention shown as a triangular geometry.

### DETAILED DESCRIPTION OF INVENTION

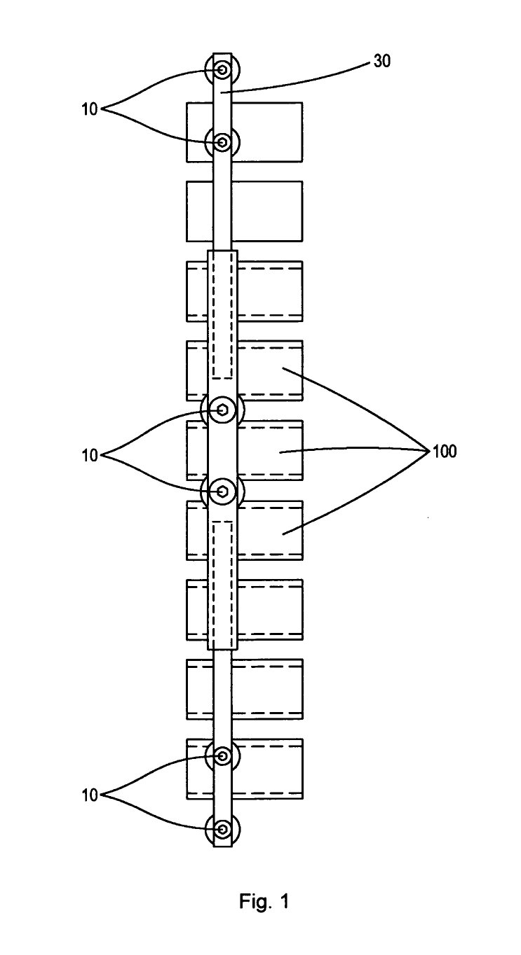

With reference to FIGS. 1-5 the present growing rod spinal deviation correction system includes a number of pedicle screws 10, each implanted in respective vertebrae 100 to which forces will be applied by way of a properly contoured spinal rod 30, initially to achieve a scoliotic correction in an initial surgical intervention, and thereafter to maintain the desired correction, even as the patient grows.

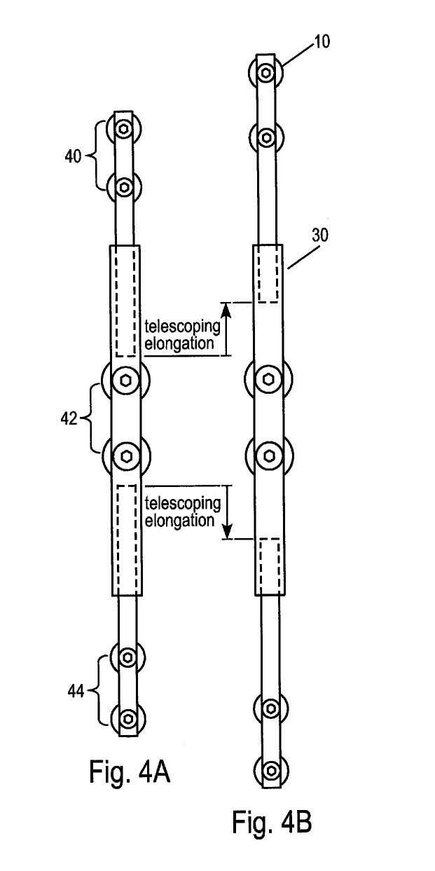

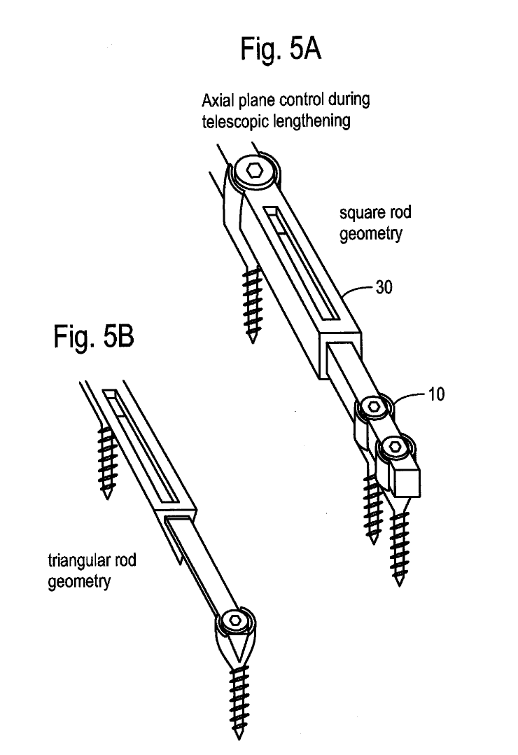

With particular reference to FIGS. 4 and 5, pedicle screws 10 and spinal rod 30 are respectively configured such that spinal rod 30 is an adjustable length spinal rod, specifically an extendable telescopic spinal rod with a means to slide or pass one end within another, may in a “slide-only engagement,” slide longitudinally with movement of the vertebrae 100 (and associated pedicle screw 10), but the same are constrained from any axial rotation and other undesirable movement because of the respective geometry of the spinal rod 30 and the portion of pedicle screws 10 with which the spinal rod 30 is mechanically linked (the “spinal rod engagement means”).

The depicted embodiment of spinal rod 30 shown in FIGS. 1, 3, 4A, 4B and 5A is of a substantially square cross-sectional geometry and in FIG. 5B of a substantially triangular cross-sectional geometry, and the associated spinal rod engagement means is configured in a complimentary fashion for both: (1) allowing longitudinal movement of the spinal rod 30 relative to pedicle screws 10 and (2) preventing axial rotation and other undesirable movement of the pedicle screw 10 relative to spinal rod 30. However, it must be understood that other “non-circular” geometries for spinal rod 30 and the rod engagement means of pedicle screws 10 may be substituted for that shown herein as a preferred embodiment. For example cross-sectional geometries (“non-circular geometries”) for spinal rods 30 may include (among others not listed) those which are triangular, hexagonal, rectangular, gear-toothed, cross-shaped, or ovoid, with the spinal rod engagement means portion of pedicle screws 10 being of a complimentary geometry. In each such case, by virtue of the relatively tight, nested engagement between a spinal rod 30 of non-circular cross-sectional geometry with a spinal rod engagement means portion of pedicle screw 10 of a complimentary geometry, substantially no axial rotation of pedicle screw 10 relative to spinal rod 30 is possible.

The extendable telescopic spinal rod 30 and pedicle screw 10 of the growing rod spinal deviation correction system may be made from any strong material such as carbon fiber or metal for long-term sustainability. Preferred materials for spinal rod 30 may be, for example, chromium or titanium or alloy thereof, more particularly cobalt chromium or cobalt chromium molybdenum or alloy thereof, or other material known to one of skill in the art.

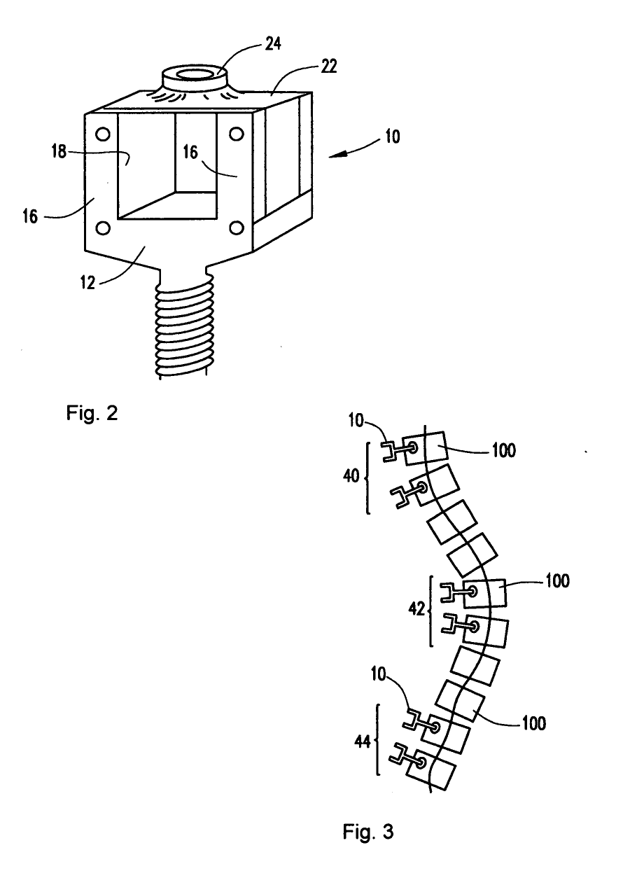

In the preferred embodiment of the pedicle screws 10 of the present invention, the head portion 12 of pedicle screws 10 is configured as a yoke-like structure for achieving a spinal rod engagement means, as depicted in FIG. 2. Two, upwardly projecting arms 16 cooperatively form this structure, defining a rod enclosure space 18, itself having a lateral opening 20 through which a segment of spinal rod 30 may be laterally introduced into the rod enclosure space 18.

A snap-fit clip 22 serves to occlude opening 20 and thereby constrain the associated length of spinal rod 30 within space 18. A set screw 24 is provided for clip 22 for use in instances where longitudinal movement of pedicle screws 10 relative to spinal rod 30 is to be prevented. In such cases, set screw 24 is adjusted in such a manner that it engages the adjacent surface of spinal rod 30 whereby substantially all relative movement between spinal rod 30 and pedicle screw 10 is arrested.

Referring particularly to FIGS. 1 and 3, the preferred method for use of the present growing rod system involves, by way of an example involving a right thoracic curve, placing pedicle screws 10 in three clusters. An upper cluster 40 involves two pedicle screws 10 placed in vertebrae 100 above the upper end vertebrae (“UEV” in FIG. 3) of the scoliotic curve; a middle cluster 42 placed in vertebrae 100 substantially at the apex of the scoliotic curve; and a lower cluster 44 placed in vertebrae 100 below the lower end vertebrae (“LEV” in FIG. 3) of the scoliotic curve. In certain embodiments, the upper cluster 40 and lower cluster 44 may serve as counter-rotation anchor points when the middle cluster 42 anchors the principal curve straightening and vertebral derotation correction.

Once spinal rod 30 is engaged with pedicle screws 10, and the initial three-dimensional scoliotic correction is achieved, clips 22 are engaged with each of the pedicle screws 10, set screws 24 of pedicle screws 10 are tightened to “anchor” spinal rod 30, while the extendable telescopic spinal rod allows the earlier-described longitudinal movement of the spinal rod with the vertebrae and associated pedicle screws 10. Accordingly, as the spinal column grows or the distance in spinal segments increases, the extendable telescopic spinal rod elongates in the same plane relative to the movement of the vertebrae and associated pedicle screws, providing for relatively uninhibited growth of the spinal segments.

The extendable telescopic spinal rod 30 of the growing rod spinal deviation correction system provides a large rod-rod sliding (extendable telescoping rod) smooth surface area interface, greatly improving the operability of the adjustable rod. The larger surface area interface for the extendable telescopic spinal rod 30 also reduces the chance for wear of the system parts, particularly metal wear and scoring that could lead to binding and possible metal debris and ion release. Metal wear and binding may occur in particular in systems in which pedicle screws are engaged to slide longitudinally along a spinal rod.

Once the present spinal rod system is implanted, as described, a juvenile patient's subsequent growth is unhindered by the system, while correction of the scoliotic curve is maintained to maturity and thereafter. Proper relative alignment of the vertebrae is maintained, as is the individual orientation of affected vertebrae, thereby achieving and maintaining a true three-dimensional scoliotic correction. Further or future spinal longitudinal growth is modulated by control in three dimensions.

Although the invention has been described with reference to specific embodiments, this description is not meant to be construed in a limited sense. Various modifications of the disclosed embodiments, as well as alternative embodiments of the inventions will become apparent to persons skilled in the art upon reference to the description of the invention. It is, therefore, contemplated that the appended claims will cover such modifications that fall within the scope of the invention.

1. An improved spinal rod system comprising:
an extendable telescopic spinal rod having a non-circular cross-sectional geometry;
a plurality of pedicle screws, each said pedicle screw having spinal rod engagement means for allowing longitudinal movement of said spinal rod, while resisting axial rotation of said pedicle screw relative to said spinal rod; and
spinal rod securing means configured for interfacing with said pedicle screw and thereafter for securing a mechanical engagement between a said segment of said spinal rod and said spinal rod engagement means.

2. The system of claim 1 wherein said spinal rod engagement means comprises constituents of a head portion of said pedicle screw which define a spinal rod enclosure space which is contoured in such a manner as to engage positively a segment of said spinal rod in a manner for preventing relative movement of said pedicle screw and said spinal rod in all directions other than substantially along the longitudinal axis of said spinal rod.

3. The system of claim 2 wherein said spinal rod exhibits a non-circular cross-sectional geometry, and said spinal rod engagement means of each said pedicle screw defines said rod enclosure space of a complimentary geometry to said spinal rod.

4. A method for correcting and maintaining correction of a scoliotic curvature of the spine comprising the steps of:
selecting a growing rod spinal rod system comprising:
an extendable telescopic spinal rod having a non-circular cross-sectional geometry;
a plurality of pedicle screws, each said pedicle screw having spinal rod engagement means for allowing longitudinal movement of said spinal rod, while resisting axial rotation of said pedicle screw relative to said spinal rod; and
spinal rod securing means configured for interfacing with said pedicle screw and thereafter for securing a mechanical engagement between a said segment of said spinal rod and said spinal rod engagement means;
implanting a first said pedicle screw in an upper vertebra;
implanting a second said pedicle screw in a middle vertebra;
implanting a third said pedicle screw in a lower vertebra;
aligning a plurality of vertebrae to achieve a scoliotic correction;
engaging said spinal rod with said spinal rod engagement means respectively of the first, second and third said pedicle screws; and
engaging said spinal rod securing means to each of the first, second and third said pedicle screws.

5. The method of claim 4 wherein said spinal rod engagement means comprises constituents of a head portion of said pedicle screw which define a spinal rod enclosure space which is contoured in such a manner as to positively engage a segment of said spinal rod in a manner for substantially preventing relative movement between said pedicle screw and said spinal rod in all directions other than substantially along the longitudinal axis of said spinal rod.

6. The system of claim 5 wherein said spinal rod exhibits a non-circular cross-sectional geometry, and said spinal rod engagement means of each said pedicle screw defines said rod enclosure space of a complimentary geometry of said spinal rod.
